(19) United States
(12) Patent Application Publication
(10) Pub. No.: US 2021/0022589 A1
(43) Pub. Date: Jan. 28, 2021

(54) CONTAINER FOR TREATMENT OF ENDOSCOPES

(71) Applicant: CISA PRODUCTION S.R.L., Lucca (IT)

(72) Inventor: Francesco CROTTI, Lucca (IT)

(21) Appl. No.: 16/755,568

(22) PCT Filed: Oct. 10, 2018
(86) PCT No.: PCT/IT2018/050190

§ 371 Date: Apr. 11, 2020

(30) Foreign Application Priority Data
Oct. 13, 2017 (IT) 102017000115546

(51) Int. Cl. A61B 1/00; A61B 1/12; A61L 2/26

(52) U.S. Cl. CPC A61B 1/00144; A61B 1/121; A61L 2202/24; A61L 2202/17; A61L 2202/18; A61L 2/26

(57) ABSTRACT

A container for endoscopes, usable for the containment of endoscopes during washing, sterilization, drying and for the subsequent storage of endoscopes subjected to such operations, characterized in that it is provided with signaling means (7) applied to said container (2) and adapted to emit a signal if the pressure inside the same container (2) is below a pre-established value.

The present invention relates to a container for washing, disinfecting/sterilizing and storing endoscopes. In particular, the container of the invention allows washing, disinfection, sterilization and drying of endoscopes inside the container in which the same instrument can then be stored.

It is known that the use of endoscopes requires frequent washing and disinfection operations due to the daily use of the same endoscope on several patients. On some of these instruments the sterilization is required, which involves further steps, among which the packaging and the treatment inside a low temperature sterilizer.

Traditional multi-stage procedures are known which provide for the execution of washing, disinfection and sterilization in different phases and/or operating stations; in practice, with these systems it is not possible to perform a single treatment cycle to wash, disinfect, sterilize and package an endoscope using a single operative process. The disadvantages of these systems and the consequent deficiencies in terms of safety and functionality are self-evident.

A solution to the aforementioned drawbacks was provided by EP1696969B1 which discloses a system for the washing, disinfection, sterilization, drying and storage of endoscopes which provides for the use of a hermetically sealed container, in which a containment compartment for an endoscope is formed. This container is provided with a multiple connector that allows the connection of the endoscope channels with a system of external pumps through which washing and sterilization fluids are fed into the same channels. The multiple connector also allows to feed a gas for emptying or drying said channels. The system, in addition to the container, consists of two machines, the first for washing, disinfection, sterilization, the second for hot air drying.

The present invention relates to a container of the type described above and it is intended to further increase its effectiveness and ease of use, providing further advantages both from the safety point of view and from the construction point of view relating to the construction of the apparatus and the container.

This result has been achieved, in accordance with the present invention, by adopting the idea of realizing a container having the characteristics indicated in claim 1. Other features of the present invention are the subject of the dependent claims.

A container for washing, sterilizing and storing endoscopes according to the present invention has improved utilization characteristics, resistance to stress induced by the washing cycles, sterilization and storage, as well as improved safety features. Among the advantages of the present invention, the following can be listed in a non-limiting way: the container can be equipped with at least two types of covers, so as to be able to contain endoscopes of different sizes, reducing the consumption of washing and/or sterilization fluids in the case of the configuration with the lower volume; the connection between the base and the cover includes a sealing gasket to make the container impermeable to fluids, including air; the container has a bilobed shape in diametrical section, that is axial symmetric, so as to define a smaller internal volume with respect to a corresponding container with non-concave walls and to guarantee a considerable resistance to pressure, allowing cleaning, sterilization and drying treatments even when empty, and the conservation of the endoscope at a pressure greater than the atmospheric pressure; the container can advantageously be provided with an indicator of the internal pressure able to indicate anomalies related to pressure values lower than a predetermined alarm value; the shape of the container and the presence of a support allow its vertical positioning, both inside a sterilizing machine, and during storage; the container is provided with at least two openings for the connection to the sterilizer machine so as to optimize the washing and sterilization processes, with an easy drainage, also thanks to its conformation mentioned previously; the preferential arrangement of the two connection openings, respectively at the top and at the bottom, allows a bottom-up filling of the container with the complete immersion of the endoscope, substantially reducing to zero the possibility of non-immersed portions of the endoscope and ensuring a constant upward exit of air; the preferential presence of at least one flow diverter inside the container allows to create a movement of the washing fluid capable of increasing the efficiency of the treatment; the container can advantageously be provided with an integrated handle to improve the ease and efficiency of the transport, as well as its handling during insertion and extraction from treatment and/ or storage apparatuses; the container can advantageously be provided with a radio frequency or RFID type identification element which allows its unambiguous identification; the container of the present invention can be configured to allow flow and pressure control independently in a machine arranged for this purpose; moreover, the same container does not preclude its use in a sterilizing machine which utilizes a “trough the wall” passage, i.e. a passage between two zones with a different degree of sterility. These and further advantages and characteristics of the present invention will be better understood by any person skilled in the art, thanks to the following description and to the attached drawings, provided as an example but not to be considered in a limitative sense, in which:

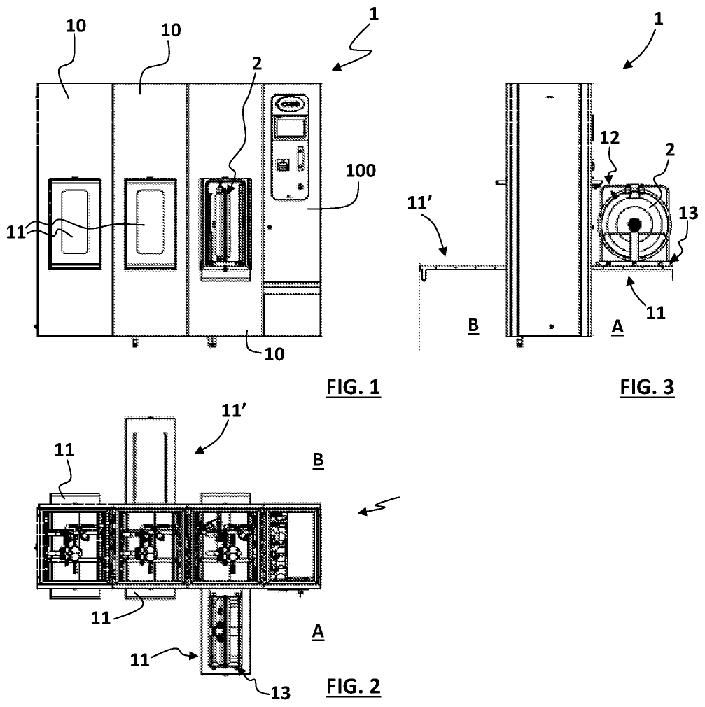

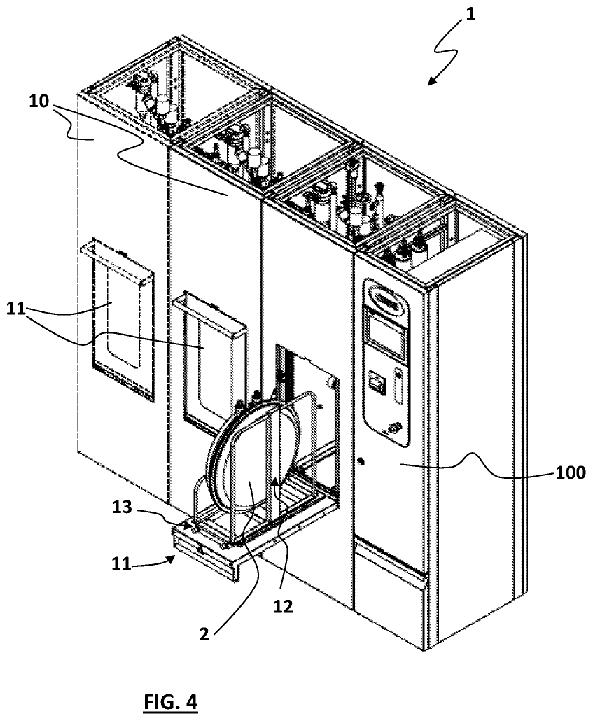

FIGS. 1, 2, 3 and 4 relate to a possible embodiment of an apparatus for washing, sterilizing and drying endoscopes, represented, respectively, in a front view (FIG. 1), in a plan view from above (FIG. 2), in a side view (FIG. 3) and in a perspective view (FIG. 4);

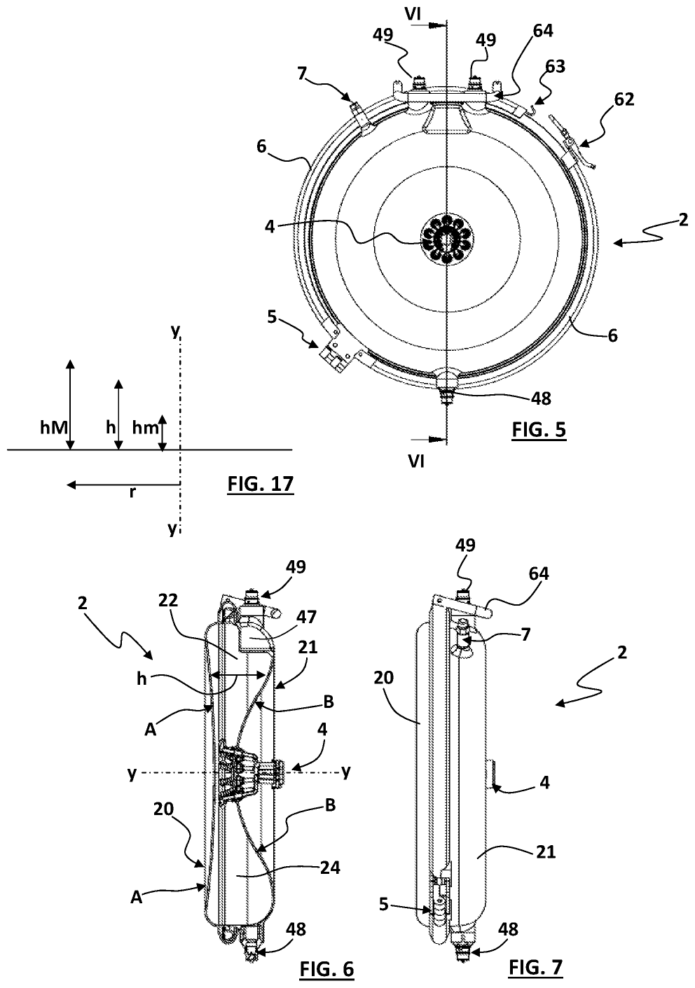

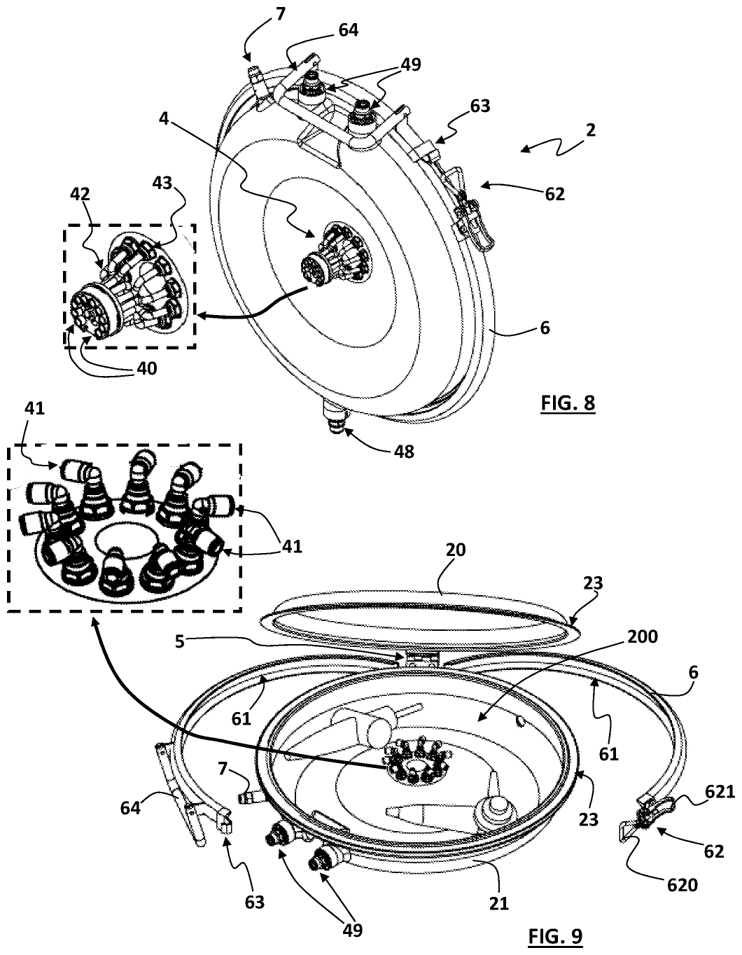

FIGS. 5, 6, 7, 8 and 9 relates to a possible embodiment of a container for the washing, sterilization and drying of endoscopes according to the present invention, represented respectively in a front view (FIG. 5), in a section view according to the line VI-VI of FIG. 5 (FIG. 6), in a side view (FIG. 7), in a perspective view with an enlarged detail (FIG. 8) and in a perspective exploded view with an enlarged detail (FIG. 9);

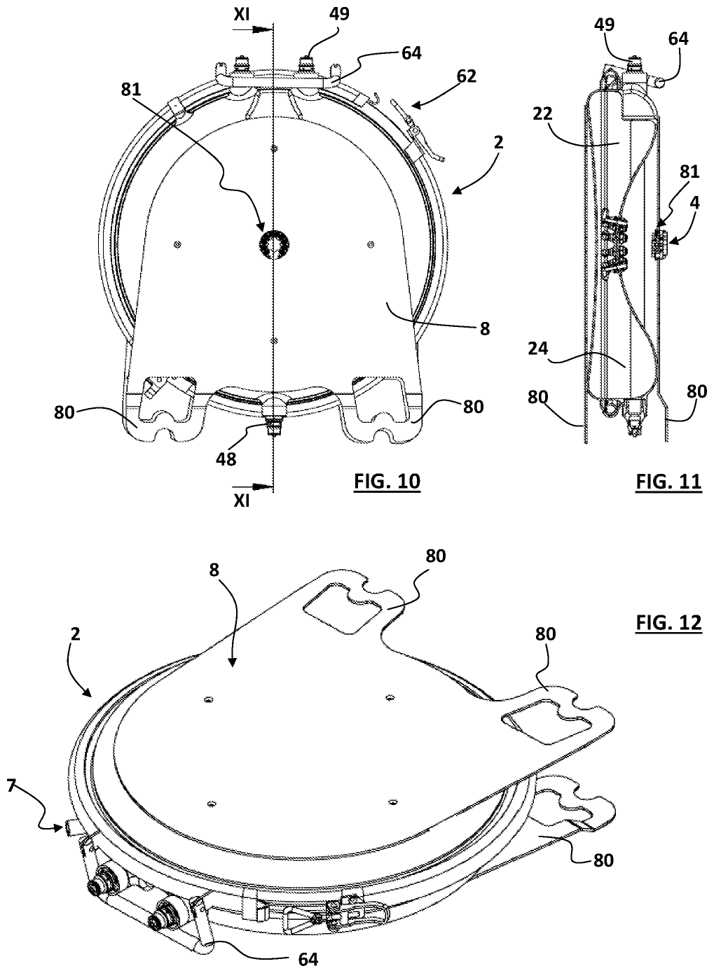

FIGS. 10, 11, and 12 relate to the embodiment of the container shown in FIGS. 5-9 provided with a supporting structure, the container being represented respectively in a front view (FIG. 10), in a section view according to the line XI-XI of FIG. 10 (FIG. 11), and in a perspective view (FIG. 12);

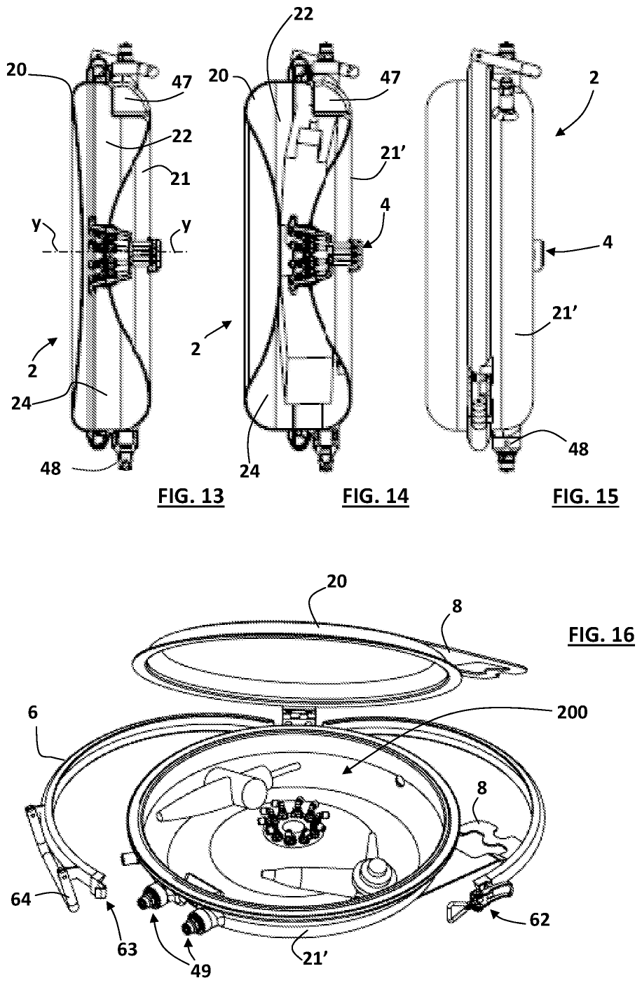

FIG. 13 corresponds to FIG. 6 and it is placed near to FIG. 14 to better show the differences between the example of FIGS. 5-9 with the embodiment of FIGS. 14, 15 and 16, which relate to another possible embodiment of a container according to the present invention, in particular for an instrument of greater dimensions such as an eco-endoscope; the container being represented, respectively, in a diametrical section view (FIG. 14), in a side view (FIG. 15) and in an exploded perspective view (FIG. 16);

FIG. 17 relates to a diagram that indicates the progression of the internal height of the container according to the distance from the center of the same container. Reduced to its essential structure and with reference to the attached exemplary drawings, a container (2) for the washing, sterilization and storage of endoscopes according to the present invention has a substantially circular profile and comprises two complementary and separable parts (20, 21; 21′) designed to delimit an internal compartment (200) in which an endoscope can be positioned. In the present description, one of said two parts will be called base (20) and the other cover (21). On the base (20) a multi-connector (4) is mounted in order to allow, as further indicated below, to connect the endoscope channels with a system of external pumps through which washing and disinfecting/sterilizing fluids are fed into the same channels. Said pumps are supported by a suitable washing, sterilization and drying apparatus (1) provided with means for its connection to the multi-connector (4). The multiple connector (4) also allows to feed, by means of corresponding connection means presented by said apparatus (1), a gas for emptying or drying said channels. Said connector (4) comprises a plurality of external couplings (40) each of which is connected to a respective internal bush (41) by means of a corresponding tube (42) and relative fittings (43). The external couplings (40) are provided on the external side of the part (21), while the bushes (41) are on the inner side of the same part (21). In practice, through the multi-connector (4) the apparatus (1) is able to perform the washing, sterilization and drying of the internal parts of the endoscope in a safe, rapid and effective manner. The structure of the multi-connector (4) is known per se.

The container (2) can be formed by the association of the base (20) with differently sized covers (21, 21′). With particular reference to FIGS. 13-16, it is possible to identify a kit, that is a set of elements comprising a base (20) and at least two covers (21, 21′). In this way the container (2) can be equipped with at least two types of covers, so as to be able to contain endoscopes of different sizes (for example simple endoscopes and eco-endoscopes), reducing the consumption of washing and/or sterilization liquids in the case of configuration with the smaller volume. In practice, the cover (21) having higher height allows to define a larger volume (200), while the other cover (21′) allows to define a smaller volume (200), always using the same base (20).

The association between the base (20) and the cover (21) can be carried out in various ways, for example by means of hinges and circular clamps. In FIG. 5 a circular clamp (6) is shown, formed by two substantially semi-circumferential parts fixed at a first end to a hinge and provided at the other end, or distal end, with a stable connection means, which in the example is constituted by a hook element (63) and a corresponding lever element (62) allowing the container (2) to be closed.

The base (20) and the cover (21) can be provided with corresponding peripheral edges on which the circular clamp (6) is fitted, preferably with a sealing gasket, the clamp having an internal profile complementarily shaped with respect to said edges. In practice, the closure of the container (2) provides for the approach of the base and the cover, the covering of the edges (23) through the two parts of the clamp (6) and the locking of the latter thanks to the interaction between the open part (620) of the lever element (62) with the hook element (63) and the activation of the lever (621) of element (62). The connection so realized between the base and one of the covers makes the container (2) impermeable to fluids, including air, making the positioning of the endoscopes inside the containers as well as their subsequent pickup safe, easy and effective. Obviously the materials chosen for the construction of the clamp (6) and its parts will be suitable to allow a stable closure. The circular clamp (6) is also provided with a handle (64) which, in the illustrated examples, is pivoted to the body of the clamp itself, being hinged about an axis which is substantially tangential with respect to the circular profile of the clamp (6).

The handle (64) may also be made differently as shown in the schematic example of FIG. 6. In addition to the multi-connector (4), the container (2) is also provided with openings to allow the fluids to entry and exit the container. In the drawing, two openings (49) are provided in the area of the container (2) which in use will be arranged on top, while an opening (48) is disposed in the diametrically opposed zone, that is, the zone that in use will be placed at the bottom. Inside the container (2), for example (but not exclusively) in correspondence of the openings, means can be advantageously provided for diverting the fluids at the inlet to increase the efficiency of the treatment; in the drawings, the diverting means are schematically represented by a diverter (47) formed by a particular internal shape of the cover (21; 21′) that, when is associated with the base (20), forms an inclined surface in correspondence of the opening (49). Through the aforementioned openings it is possible to fill (and empty) the inside of the container (2) to allow cleaning/sterilization/ drying of the external parts of the endoscope and the internal parts of the container. Particularly relevant is also the shape of the container (2) which, seen in diametrical section, has a bilobed shape (for example, the diametrical section shown in FIG. 11 corresponds to a sectional view of the container 2 formed along the diameter XI-XI of the same container whose outer profile is circular; similarly, the diametrical section shown in FIG. 6 corresponds to a sectional view along the diameter indicated by VI-VI in FIG. 5); in practice, the container has an axially symmetrical conformation with respect to a central axis (y-y) and has a convex internal wall developing symmetrically with respect to said axis, so as to guarantee a considerable resistance to pressure, allowing cleaning and sterilization treatments even when empty; as well as the storage of the endoscope at a pressure greater than the atmospheric pressure. Moreover, this conformation defines a smaller internal volume with respect to a corresponding container with non-concave walls. In the drawings, to better highlight the characteristic shape of the container (2), the lobes which are defined inside it have been marked with (22) and (24); the indication and differentiation of the lobes has the purpose of better highlighting the bilobed conformation even if the two lobes (22, 24) are actually joined together to form the whole internal volume (200) of the container (2). In other words, the container (2) develops symmetrically with respect to the axis of symmetry (y-y) and the surface of its inner walls delimits the aforementioned internal volume (200).

In the context of the present description, a diametrical section plane means a section plane which contains an axis of symmetry of the container which, with reference to the example shown in the drawings, is the axis (y-y) passing through the center of the multi-connector (4), the latter being placed in a central position in the container (2). As previously stated, a diametrical section plane is indicated by line VI-VI of FIG. 5. Moreover, in the context of the present description, by bilobate shape it is meant a shape comprising two lobes (22, 24) opposed to the aforesaid axis (y-y). In practice, in any cross-sectional or diametrical cross-section plane (VI-VI), the parts (22, 24) of the container (2) opposed to the axis (y-y) have the shape of lobes, such that their height (h) is not constant but it changes with the distance from the same axis (y-y), being smaller in proximity to the latter. For example, with reference to the example illustrated in FIG. 6, the wall (A) of the base (20) of the container (2) is concave, with the concavity facing the outside of the container, i.e. with the convexity facing towards the internal of the latter; and the inner side of the upper wall (B) or cover (21), seen in section in the aforesaid plane (VI-VI), is convex near the axis (y-y) and is concave near the edge of the container. In practice, the container (2) is flattened in its central part with respect to its periphery. As schematically illustrated in FIG. 17, the height of the container (2) varies according to the radius (r), i.e. the distance from the axis (y-y), of the point where the height is measured. In FIG. 17 the reference (hm) represents a minimum value of the height of the container (2), while the reference (hM) indicates a maximum height value and the reference (h) indicates the height of the container (2) at a generic radial distance (r) from the axis (y-y). The minimum value (hm) is greater than zero.

Therefore, an endoscope container, usable for the containment of endoscopes during the washing, sterilization, drying and the subsequent storage of endoscopes subjected to such operations, according to the accompanying drawings, is an axial symmetric hollow body with an axis of axial symmetry (y-y), defining an internal volume (200) having a bilobed cross section, with two lobes (22, 24) symmetrical with respect to said axis (y-y) and whose height (h) measured parallel to the same axis (y-y) varies between a maximum value (hM) and a minimum value (hm) as a function of the distance (r) from the latter.

With reference to the example shown in FIGS. 5-16, the container (2) is circular in shape. The container (2) can advantageously be provided with an internal pressure indicator (7) capable of indicating anomalies corresponding to pressure values lower than a predetermined alarm value. The indicator (7) can consist of a pressure gauge or an indicator with an index that moves proportionally to the internal pressure, or any other pressure meter. It can also emit visual and/or acoustic signals.

The indicator (7) is able to signal possible anomalies when the container (2) has left the treatment station in which it has been subjected to at least one washing, disinfection/ sterilization and drying treatment and where the pressure inside it has been brought at a value higher than the atmospheric pressure. The indicator (7) indicates the possible lowering of the pressure inside the container (2) below a predetermined limit value. This lowering of the pressure in the container (2) could be due, for example, to an accidental breaking of the container or to an unauthorized opening of the container itself.

According to the accompanying drawings, said indicator (7) is integral with the container (2). In other words, the indicator (7) is applied on the container (2). Therefore, the indicator (7) continues to perform its function even when the container (2) is removed from the apparatus (1) through which the same container is internally pressurized. Consequently, it is possible to check whether the container is internally pressurized even when it, once disconnected from the apparatus (1), is placed in a storage area where the operators can pick it up. In other words, since the indicator (7) is integral with the container (2), it is possible to check whether the internal pressure of the latter has fallen below the predetermined value at any time between the completion of the operations performed by the apparatus (1) and the use of the container (2).

This feature is particularly important because it allows to have a substantially error-free indicator of the sterility preservation. In other words, at the end of the washing, sterilization and drying treatments, which may take place in the apparatus (1), it is possible to pressurize the inside of the container (2), that is, to bring the pressure inside the container to a value higher than that atmospheric, and then monitoring the internal pressure value so as to provide an alarm signal through the indicator (7). For example, the indicator (7) can consist of a pressure gauge or it can be connected to a pressure gauge inside the container (2). It can also emit visual and/or acoustic signals. It is sufficient that the indicator (7) is able to assume two configurations, i.e. a configuration of normality and an anomaly configuration; in other words, it is possible to use a device capable of emitting a signal when the detected pressure goes beyond a predetermined pressure value inside the container (2).

In view of above, it is understood that the present invention starts from an assumption which is opposite to systems which provide for the maintenance of sterilized instruments at the atmospheric pressure, isolated from the outside only by means of containers provided with filters or special packaging or casings of a known type. In fact, a possible loss in a container or the damage to a packaging causes the entry of fluids inside it with the possibility of introducing also pathogenic germs and similar particles that are not allowed; on the contrary, the natural loss of pressure in a pressurized container would imply the escape of gas, avoiding the entry of external agents, maintaining the sterility condition inside it until the achievement of an equality between the external and internal pressures. The indicator (7) therefore allows to visualize the safety condition corresponding to a pressure inside the container greater than the atmospheric pressure and, vice versa, the non-safety condition corresponding to a pressure inside the container lower than the atmospheric pressure.

Another feature of the present invention relates to the possible presence of a support structure (8) of the container (2) capable of allowing vertical positioning of the container (2) while maintaining the axial-symmetric conformation of the container (2) without varying the shape of the latter. The support structure (8) is formed by two plates that can be fixed to the two sides of the container (2). The plates (8) are provided with supporting feet (80) and one of them is provided with a perforated portion (81) suitable for allowing the multiple connector (4) to pass through it. In practice, the structure (8) allows the container (2) to be arranged vertically, allowing access both to the multiple connector (4) and to the upper (49) and bottom (48) openings.

The structure (8) and the handle (64) make the handling of the container extremely simple and safe for the operators.

As previously stated, the washing, disinfection/sterilization and drying treatment can be carried out using an apparatus (1) such as that shown by way of example in FIGS. 1-4. The apparatus (1) has a structure comprising one or more modules (10) suitable for receiving the container (2). Each module is provided with a pair of doors (11, 11′) hinged on a horizontal axis so as to be positioned on at least one closing configuration, in which the door (11, 11′) is substantially vertical, and on a opening configuration, in which the door (11, 11′) is substantially horizontal, forming an approximately 90° angle with the vertical wall of the apparatus (1).

One (11) of the doors (11, 11′) is placed upstream, or in an area, marked with (A) in the drawings, where a certain degree of sterility is expected, while the other door (11′) is placed downstream, or in an area, marked with (B) in the drawings, where a different degree of sterility is expected, usually higher than that of zone (A). In other words, a passage called “trough the wall” is realized, that is a passage between two zones with a different degree of sterility.

Each door (11, 11′) is provided with guides (13) suitable for housing the container (2), preferably but not exclusively provided with the support structure (8). In addition or alternatively to the guides (13), a guide structure (12) can also be provided, also designed to house the container (2).

During treatment, a container (2) with an endoscope inside it is placed on the upstream door (11) that has been previously brought into the opening configuration. The downstream door (11′) is closed. The container (2) is then introduced into the apparatus (1) where appropriate connectors connect the operative components of the apparatus such as pumps, sensors, etc. to the corresponding connection means (4, 48, 49) of the container (2). The upstream door (11) is then closed.

The apparatus (1) provides, using the multiple connector (4), to perform in succession the internal washing treatment of the endoscope, its sterilization and subsequent drying.

Similarly, through the openings (48, 49), the apparatus (1) performs the external treatment of the endoscope and of the inside of the container (2).

The container (2) can be made of plastic material, composite material or any other material suitable for the purpose.

Advantageously, the apparatus (1) can be used for the sterilization of endoscopes and similar instruments, and is of the type comprising connecting means for a container inside which the endoscope to be sterilized is placed, said means being suitable for introducing into said container one or more washing and/or sterilizing fluids and one or more fluids for drying the endoscope. In practice the apparatus not only sterilizes, but also performs the washing and drying of the endoscopes inside the container (2). The use of this apparatus does not require sterile packaging operations that instead characterize the systems that are not able to perform the complete treatment of the instrument.

The apparatus (1) is provided with means for pressurizing said container (2), that is to pressurize it at a pressure higher than the atmospheric pressure. The means for carrying out the pressurization can be pumps or other means suitable for this purpose.

The container is brought to a pressure higher than the atmospheric pressure. In particular, the pressure inside the container (2) is preferably brought to a value of 250-350 mbar (250-350 hPa, i.e. 25000-35000 Pa) higher than the atmospheric pressure i.e. higher than the atmospheric pressure of the environment in which the pressurized container (2) is stored. With the apparatus (1) it is therefore possible to carry out a method for the sterilization of endoscopes and similar instruments, of the type comprising the washing and/or disinfection/sterilization of an endoscope inside a container by one or more washing fluids and/or the subsequent drying by one or more drying fluids. The method is characterized by the fact that the container (2) is pressurized, that is, by the fact that inside it a pressure higher than the atmospheric pressure is provided at the end of the drying phase, i.e. when the container is released with the sterilized and dried instrument inside it. The apparatus (1), again by means of the aforementioned connections (4, 48, 49) and its components suitable for the purpose, is provided with means capable of determining a pressure inside said container (1) considerably lower than atmospheric, said pressure being apt, in addition, to increasing the drying efficiency and reducing drying times. The means for carrying out the depressurization can be pumps or other means suitable for the purpose. A method and an apparatus usable with a container (2) according to the invention can determine inside the container a pressure considerably lower than the atmospheric pressure, for example less than 20 mbar or less than 10 mbar, or even 1 mbar (respectively corresponding to 2000 Pa, 1000 Pa, and 100 Pa).

In this way the drying time is drastically reduced.

Depressurization is also advantageous in the sterilization phase because, thanks to the depressurization, the sterilizing agent diffuses in the container (2) more effectively. The values indicated are not limiting of other embodiments of the invention. Moreover, the execution details may in any case vary in the form, dimensions, arrangement of the elements, nature of the materials used, without however departing from the scope of the idea of solution adopted or of the inventive concept and therefore remaining within the limits of the protection granted by the present patent.

1. A container Container for endoscopes, usable for the containment of endoscopes during washing, sterilization, drying and for the subsequent storage of endoscopes subjected to such operations, characterized in that it is provided with signaling means applied to said container and adapted to emit a signal if the pressure inside the same container is below a pre-established value.

2. The container Container for endoscopes, according to claim 1, wherein said signaling means are of visual and/or acoustic type.

3. The container for endoscopes, according to claim 1, wherein said container comprises two complementary and separable parts suitable for defining an internal compartment in which an endoscope can be positioned, one of the parts or base being provided with a multiple connector which allows to connect the channels of the endoscope with a system of external pumps through which washing and sterilization fluids are fed into the same channels.

4. The container for endoscopes, according to claim 1, wherein said container comprises a plurality of openings to allow the entry and exit of fluids inside the container.

5. The container for endoscopes, according to claim 1, wherein said container comprises a plurality of openings to allow the entry and exit of fluids inside the container and means for diverting the fluids at the entry opening to increase the efficiency of the treatment.

6. The container for endoscopes, according to claim 1, wherein it is provided with a supporting structure provided with supporting elements suitable for allowing vertical positioning of the container.

7. The container for endoscopes, according to claim 1 wherein it is an axial-symmetrical hollow body with an axis of axial symmetry, defining an internal volume having a bilobed cross section, with two lobes that are symmetrical with respect to said axis.

8. The container for endoscopes, according to claim 7, wherein the height of said lobes, measured parallely to the same axis, varies between a maximum value and a minimum value according to the distance from the axis.

9. A kit for making a container for endoscopes according to claim 1, of the type comprising a base and a cover which are joined together to form the said container, kit characterized in that it comprises a base and at least two covers of different profiles so as to define corresponding containers of different volumes.
